(19) United States
(12) Patent Application Publication
(10) Pub. No.: US 2025/0143794 A1
(43) Pub. Date: May 8, 2025

(54) SYSTEM FOR MONITORING TEMPERATURE WHILE INTRACORPORAL LASER LITHOTRIPSY IS BEING CARRIED OUT

(71) Applicant: GYRUS ACMI, INC. D/B/A OLYMPUS SURGICAL TECHNOLOOGIES AMERICA, Westborough, MA (US)

(72) Inventors: Arkadiusz Miernik, Freiburg (DE); Simon Hein, Freiburg (DE); Ralf Petzold, Freiburg (DE)

(21) Appl. No.: 18/982,821

(22) Filed: Dec. 16, 2024

Related U.S. Application Data
(63) Continuation of application No. 17/625,801, filed on Jan. 10, 2022, now Pat. No. 12,193,737.

(30) Foreign Application Priority Data
Jul. 11, 2019 (DE) 202019103823.3

(51) Int. Cl. A61B 18/26; A61B 18/00

(52) U.S. Cl. CPC A61B 18/26; A61B 2018/00005; A61B 2018/00648; A61B 2018/00678; A61B 2018/00702; A61B 2018/00732; A61B 2018/00791; A61B 2018/00863; A61B 2018/0094; A61B 2018/00982; A61B 2218/002

(57) ABSTRACT

A system for monitoring temperature when carrying out laser light-based lithotripsy in which includes an endoscopic assembly comprising a working channel for a fiber optic cable which is optically coupled to a laser on a proximal side and include a light exit aperture on a distal side, and an irrigation fluid channel opening into a region of the light exit aperture on the distal side which is in fluid communication with an irrigation fluid reservoir on the proximal side. The system includes a modular unit including a flow sensor, which determines the irrigation flow rate without corning into contact with the irrigation fluid; input, via which operating parameters of the laser can be determined, can be transmitted to a processor connected to the input; a temperature sensor which determines the temperature of the irrigation fluid without corning into contact with the irrigation fluid; an analyzer, which numerically determines the temperature of the irrigation fluid and the determined applied laser power, and the temperature generated intracorporeally during the laser lithotripsy at the location of the light exit aperture, and a comparator, which produces a signal in the event that a threshold value is exceeded.

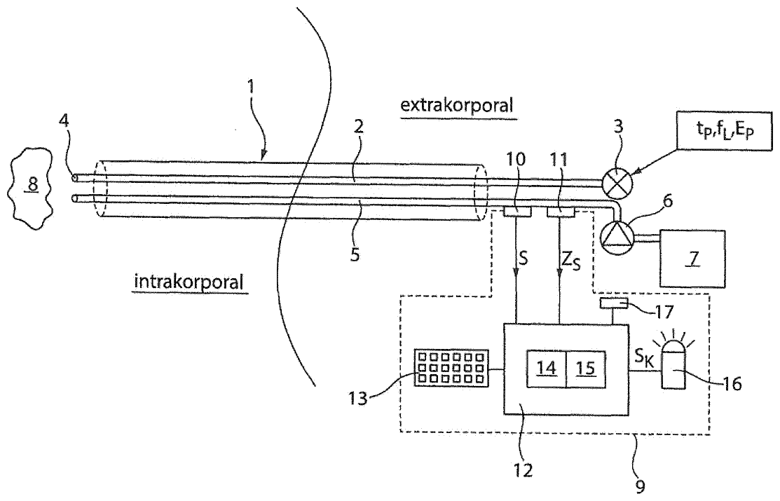

### PRIORITY CLAIM

This application is a Continuation of U.S. patent application Ser. No. 17/625,801, filed Jan. 10, 2022, which is a National Stage Entry of PCT/EP2020/069125, filed Jul. 7, 2020, which claims priority to German Application DE 20 2019 103 823.3, filed Jul. 11, 2019, the contents of which are hereby incorporated by reference.

### TECHNICAL FIELD

The invention relates to a system for monitoring temperature when carrying out laser light-based intracorporeal lithotripsy, in which an endoscopic assembly is employed which comprises a working channel for a fibre optic cable, which is optically coupled to a laser on the proximal side and has a light exit aperture on the distal side, as well as an irrigation fluid channel which opens into the region of the light exit aperture on the distal side and is in fluid communication with an irrigation fluid reservoir on the proximal side.

### PRIOR ART

Intracorporeal lithotripsy is a minimally-invasive surgical method for shattering stone concretions, for example gallstones, urinary stones or kidney stones, which primarily collect in excretory ducts in the affected organs and can cause severe pain as well as other medical problems. Of the many known intracorporeal lithotripsy procedures, laser-induced lithotripsy has become an established procedure, during which energy-rich laser beams are applied via a fibre optic cable to the location of a stone to be shattered in the form of laser pulses. The interaction of the laser pulse with the intracorporeal medium forms a bubble of vapour; their pulse-like expansion generates pressure waves which lead to fragmentation of the stone. Depending on their size, the fragments of stone generated by laser-induced lithotripsy can be excreted naturally or they can be extracted with suitably configured endoscopic gripping tools.

Endoscopes are used to carry out laser-based lithotripsy; they have at least one working channel for a fibre optic cable which is optically coupled to a laser on the proximal side and have a light exit aperture on the distal side. In order to be able to follow the organic access routes to the stones with the endoscope and to burden patients as little as possible during the treatment, endoscopes of this type may be pliable and flexible in configuration. In addition, an irrigation fluid can be applied via endoscopes of this type, usually via a working channel which opens into the region of the light exit aperture on the distal side and is in fluid communication with an irrigation fluid delivery device on the proximal side, preferably in the form of an irrigation syringe and/or a controllable or regulatable delivery pump, as well as with an irrigation fluid reservoir.

With the aid of an endoscopic assembly of this type comprising the endoscope and the aforementioned peripheral devices which are connected to it, the laser power applied intracorporeally at the location of the stone to be shattered as well as the quantity of irrigation fluid which is discharged to the irrigation channel on the distal side have to be matched to each other in a manner such that on the one hand, effective stone shattering can be carried out, and on the other hand, no local overheating of surrounding areas of tissue caused by the laser light occurs, as this could result in irreversible damage to the tissue. For the most part, endoscopes of this type are provided with additional glass fibre optics for the operator or an imaging sensor at the tip of the endoscope which has to be kept optically clear with the aid of a metered delivery of irrigation fluid in order to guarantee a clear view for the operator in order to monitor the shattering process.

The publication US 2018/0055568 A1 describes an endoscopic assembly of this type for carrying out laser-based lithotripsy with an associated irrigation fluid delivery unit which can be adjusted as a function of at least one of the parameters influencing the lithotripsy process, i.e. the irrigation flow rate is influenced as a function of the lithotripsy process. In addition to optical monitoring of the shattering process, the known endoscopic assembly is provided with a temperature sensor attached to the distal end of the endoscope which is capable of detecting the distal temperature of the fluid surrounding it, on the basis of which the irrigation fluid delivery unit is regulated in order to adjust the irrigation fluid flow rate.

Even in the case of the known medical endoscope in accordance with the publication DE 20 2017 102 316 U1, which is suitable for carrying out laser-based lithotripsy, an ambient temperature sensor is disposed at the distal region of the endoscope for the purposes of temperature measurement.

Endoscopic assemblies equipped with sensors in this manner constitute extremely complicated medical instruments which have to undergo protracted and comprehensive medico-technical certification and testing.

### DESCRIPTION OF THE INVENTION

The objective of the invention is to further develop a system for monitoring temperature when carrying out laser light-based intracorporeal lithotripsy, in which an endoscopic assembly is employed which comprises a working channel for a fibre optic cable, which is optically coupled to a laser on the proximal side and has a light exit aperture on the distal side, as well as an irrigation fluid channel which opens into the region of the light exit aperture on the distal side and is in fluid communication with an irrigation fluid reservoir on the proximal side in such a manner that it becomes possible to carry out monitored intracorporeal lithotripsy with standard endoscopes of this type without having to spend too much money and time in respect of medico-technical testing or certification. Measures are sought by means of which endoscopes, laser systems and other medico-technical devices which are already in use, such as irrigation fluid devices for passive and active irrigation which are generally suitable for laser-based lithotripsy, can be retrofitted with suitably selected components in order to be able to rule out any possible thermally induced tissue degradation in the region directly surrounding the stone shattering location.

The solution to the underlying objective of the invention is defined in claim 1. Features which further develop the inventive concept in advantageous manners form the subject matter of the dependent claims as well as the further description, in particular with reference to the exemplary embodiments.

In general, intracorporeal laser application without irrigation, i.e. without any supply of irrigation fluid to the region of the distal light exit aperture of the fibre optic cable, is only possible for short periods, i.e. less than a few seconds, otherwise without any cooling brought about by irrigation, a critical temperature of approximately 42° C. would be reached and exceeded, beyond which the surrounding tissue would be irreversibly heat-damaged. As a result, irrigation during laser light-based lithotripsy is indispensable in order to guarantee sufficient cooling.

A reliable numerical or theoretical relationship between the intracorporeally applied laser power, the irrigation fluid flow rate, the temperature of the irrigation fluid and an intracorporeal temperature generated during application of the laser pulse has been found with the aid of a large number of laser-based lithotripsy experiments which have been carried out

On the basis of this discovery, in the case of known endoscopes with at least one working channel for a fibre optic cable which is optically coupled to a laser on the proximal side and with a light exit aperture on the distal side as well as comprising an irrigation fluid channel which may be separate to or integral with the working channel and which opens into the region of the light exit aperture on the distal side and on the proximal side is in fluid communication with an optional irrigation fluid delivery unit and an irrigation fluid reservoir, the opportunity arises of combining it with a modular unit in accordance with the invention in the following manner: the modular unit comprises at least one flow sensor as well as at least one temperature sensor which can respectively be releasably securely attached alongside the irrigation fluid channel and/or a supply line which is in fluid communication with the irrigation fluid channel alongside the extracorporeal region of the endoscopic assembly. In this manner, the flow sensor is constructed and disposed such that the irrigation flow rate can be determined in a manner which is without contact with the irrigation fluid. At the same time, the temperature sensor is configured and attached alongside the irrigation fluid channel and/or the supply line which is in fluid communication with the irrigation fluid channel in a manner such that the irrigation fluid temperature can be determined in a manner which is without contact.

Furthermore, the modular unit comprises either an input means via which the operating parameters for the laser can be input and can be transmitted to a processor unit connected to the input means, or an interface via which the operating parameters for the laser can be transferred directly. The operating parameters for the laser preferably include the laser power, whereupon the calculation of the laser power applied to the location of the light exit aperture can be computed via the laser pulse frequency as well as the individual pulse energy.

In addition, an analysis unit is installed in the processor unit which, on the basis of the sensor-acquired irrigation flow rate, the sensor-acquired temperature of the irrigation fluid as well as the determined intracorporeally applied laser power, numerically determines a temperature which is generated intracorporeally during the laser-based lithotripsy at the location of the light exit aperture. A comparator unit is separate to or integrated into the processor unit and compares the determined temperature with an adjustable threshold value, for example 42° C., and produces a signal if the threshold value is exceeded.

In addition, in a preferred embodiment, a signal unit is provided which is wirelessly connected to or hardwired to the comparator unit and by means of which the signal produced by the comparator unit can be made haptically, acoustically and/or visually perceptible. By means of the signal unit, the physician carrying out the lithotripsy can be made aware of potential intracorporeal overheating effects so that the lithotripsy treatment can be interrupted or other measures can be taken.

The modular unit in accordance with the invention, which is preferably configured as a retrofitting set or retrofitting kit, exclusively comprises components which can be attached in a modular manner to an endoscopic assembly which is already in use without compromising the general functionality of the endoscopic assembly. Thus, the system in accordance with the invention constitutes a safety system which supports the physician when carrying out laser-based lithotripsy by means of which the physician receives reliable and dependable information which enables the lithotripsy procedure to be carried out effectively and in a patient-friendly manner.

The only information which is required in this regard is the current temperature of the intracorporeal irrigation fluid introduced via the endoscopic assembly by means of the working channel or the irrigation fluid channel, the laser power applied at the location of the stone to be shattered, as well as the irrigation flow rate at which the irrigation fluid is introduced intracorporeally through the working channel or irrigation fluid channel at the location of the laser light application.

The sensors required for the irrigation fluid temperature and irrigation flow rate respectively constitute constructional elements which are releasably securably attached to the irrigation fluid channel and/or the supply line which is in fluid communication with the irrigation fluid channel which are applied in a region of the endoscopic assembly which is not introduced intracorporeally into the body. Thus, preferably, the sensor which determines the irrigation flow rate is a flow sensor in the form of an ultrasound sensor. A temperature sensor in the form of an infrared sensor would be suitable for acquiring the irrigation fluid temperature.

In order to determine the temperature generated at the location of the light exit aperture of the fibre optic cable, the required operating parameters which can be specified for the laser are the laser pulse frequency as well as the individual pulse energy. These operating parameters can be supplied by means of a suitably configured input means, for example a keyboard, or by means of a voice-operated input means or by means of a direct interface with the laser device of the processor unit.

The analysis unit installed in the computer unit determines the temperature T generated at the location of the light exit aperture on the basis of the following formula in the following manner:

$$T = ( 14.4 K * \frac{[ laser power [ W ] ]}{[ irrigation flow rate [ \frac{ml}{min} ] ]} ) + temp of irrigation fluid [ K ]$$

The numerical relationship between the temperature generated intracorporeally during laser-based lithotripsy at the location of the laser light application and the applied laser power, the irrigation flow rate as well as the temperature of the irrigation fluid has been confirmed in a large number of in vitro as well as ex vivo experiments.

Although the above algorithm represents a simplified formulaic relationship, the expected intracorporeal end temperatures at the location of stone shattering during the laser-based lithotripsy can be sufficiently precisely predicted with it, and so local overheating, which could lead to irreversible tissue damage in the immediate surroundings of the location for the laser-based lithotripsy, can be ruled out.

In the event that the laser unit does not have an interface for indicating the laser operation, the modular unit additionally comprises a microphone unit for detecting an acoustic signal indicating the operation of the laser which is transmitted to the analysis unit for the purposes of signal analysis which is synchronous with the actual laser operation. In this manner, during the operation of the laser, the laser produces clearly detectable acoustic signals which can be detected with the microphone unit in a time-resolved manner.

Although the physician who is responsible for carrying out the lithotripsy is the sole decision-maker when carrying out the laser-based lithotripsy, in a further embodiment, the modular unit in accordance with the invention comprises an additional emergency stop switch, i.e. in addition to the acoustic, visual and/or haptic signal-emitting signal unit; the signal can trigger an interruption to the power supply for at least the laser source.

In a further preferred embodiment, the modular unit which can be adapted to an endoscopic assembly additionally has a unit for detecting the fill level of the quantity of irrigation fluid contained in the irrigation fluid reservoir. The unit is configured as a sensor provided on the irrigation fluid reservoir and/or in the form of a software-based analysis algorithm implemented in the processor unit which determines the current fill level on the basis of the irrigation flow rate detected by the sensor and the measured time period during which a discharge of irrigation fluid occurs. The sensor signal representing the fill level or the numerically determined fill level value representing the fill level serves at least for monitoring the actual state as well as for predicting the length of time to complete emptying of the irrigation fluid reservoir or as a warning upon reaching a minimum residual quantity. The information obtained in this manner is made optically or acoustically perceptible to the physician by means of a suitable indication. The indication may be made by means of the signal unit via which the physician carrying out the lithotripsy is warned about possible intracorporeal overheating effects, or by means of an additional indication.

A further embodiment enables additional monitoring of the temperature of the supplied irrigation fluid. To this end, the temperature of the irrigation fluid (Ts) which is detected by the sensor is preferably shown as an alphanumeric value on a visual display unit. Furthermore, the measured temperature is preferably supplied to the comparator unit or a further comparator unit, whereupon in the event of the temperature of the irrigation fluid (Ts) going above or below a critical temperature value, a signal is produced which serves to warn the treating physician; as an example, in the event of the irrigation fluid becoming too cold, a warning is given in order to avoid the risk of the patient becoming hypothermic.

In a further possible embodiment, the irrigation fluid delivery unit is provided with a means with the aid of which the irrigation fluid flow rate can be adjusted as a function of the temperature T generated at the location of the light exit aperture and/or as a function of at least one operating parameter of the laser. This makes it possible to provide active regulation of the irrigation fluid flow rate for the irrigation fluid delivery unit in the form of a pump as a function of the applied laser light power and/or the calculated temperatures in the intervention area.

The system in accordance with the invention for monitoring temperature when carrying out laser light-based lithotripsy can be used in a variety of anatomical areas, for example in the bladder, ureter or renal pelvic regions. Different modes which suit the intervention may be selected which depend on the area of application. In this regard, the system offers the choice of a prostate, bladder, ureter or renal pelvic mode which are distinguished by definable warning limits and stored characteristics. In this manner, the individual risk spectrum for the various operations can be addressed.

Thus, the system can be used in any laser-based procedure in urology, in particular in laser lithotripsy, enucleation of the prostate (HoLEP, ThuLEP), laser vaporization (PVP) and interstitial laser coagulation (ILC).)

### Ways of Carrying Out the Invention, Industrial Applicability

The single figure shows an endoscope 1 with a working channel for a fibre optic cable 2 which is optically coupled to a laser 3 on the proximal side and has a light exit aperture 4 on the distal side. Furthermore, the endoscope 1 has an irrigation fluid channel 5 which opens into the region of the light exit aperture 4 on the distal side and on the proximal side is in fluid communication with a fluid delivery unit 6 as well as a fluid reservoir 7. Clearly, endoscopes are also known which combine the irrigation fluid channel 5 with the working channel for the fibre optic cable 2 in one channel. Known endoscopic assemblies of this type serve to shatter intracorporeal stones 8 during the course of laser-based lithotripsy.

The system in accordance with the invention for monitoring temperature concerns a modular unit 9 which can be combined with the extracorporeal region of the endoscopic assembly comprising the endoscope 1, the laser 3, the fluid delivery unit 6 as well as the fluid reservoir 7; in a preferred embodiment, it is composed of the following components:

Alongside the irrigation fluid supply line 5, a flow sensor 10, preferably in the form of an ultrasound sensor, is detachably secured. Similarly, alongside the irrigation fluid channel 5 or a supply line which is in fluid communication therewith, a temperature sensor 11 for detecting the irrigation fluid temperature, preferably in the form of an infrared sensor, is detachably secured. The sensor signals S, Ts produced by means of the sensors 10, 11 are transmitted to a processor unit 12 in a wireless or hard-wired manner.

Furthermore, an input means 13 is provided, preferably in the form of an interface to the laser unit for automatic operating parameter transfer, or alternatively in the form of a manually operable keyboard by means of which the operating parameters for the laser 3 can be input. For the operation of the laser 3, the laser pulse frequency fr as well as the individual pulse energy Ep can be specified by the operator These operating parameters too can be supplied via the input means 13 to the processor unit 12, on the basis of which the laser power which can be applied to the location of the light exit aperture 4 can be computed.

An analysis unit 14 is installed in the processor unit 12 and which, on the basis of the operating parameters of the laser, the irrigation flow rate S as well as the temperature Ts of the irrigation fluid detected by the sensors, determines the current temperature generated at the location of the light exit aperture 4 based on the following algorithm:

$$T = ( 14.4 K * \frac{[ laser power [ W ] ]}{[ irrigation flow rate [ \frac{ml}{min} ] ]} ) + temp of irrigation fluid [ K ]$$

Next, the determined intracorporeal temperature T is compared in a comparator unit 15 with an adjustable threshold value which is preferably TK=42° C. In the case in which the threshold value TK is exceeded, the comparator unit 15 produces a signal SK which is supplied to a signal unit 16 which can then be perceived haptically, acoustically and/or visually by the physician who is carrying out the lithotripsy.

In addition, a microphone unit 17 is connected to the processor unit 12, by means of which the activity of the laser 3 is detected; this ensures that the data processed by the processor unit 12 and the analysis unit 14 implemented therein as well as the production of a signal SK based on it is synchronous with the actual operation of the laser 3.

### LIST OF REFERENCE NUMERALS

1 endoscope
2 fibre optic cable
3 laser
4 light exit aperture
5 irrigation fluid
6 irrigation fluid delivery unit
7 irrigation fluid reservoir
8 stone
9 modular unit
10 flow sensor
11 temperature sensor
12 processor unit
13 input means
14 analysis unit
15 comparator unit
16 signal unit
17 microphone unit

1. (canceled)

2. A system for providing user perceptible feedback during a therapeutic laser procedure, the system comprising:
a temperature sensor for determining a temperature of a flushing fluid supplied to a surgical site;
a flow sensor for determining a flow rate of the flushing fluid supplied to the surgical site;
an input means for receiving a value of a parameter associated with a laser source such that a laser power can be applied at a location of a light emission aperture;
an evaluation unit for determining a temperature at the location of the light emission aperture based at least in part on the determined temperature, determined flow rate and the received value of the parameter associated with the laser source; and
a comparator unit for providing the user perceptible feedback when (i) the determined temperature reaches or exceeds a first predetermined threshold, and/or (ii) the determined temperature falls below a second predetermined threshold.

3. The system of claim 2, wherein the flow sensor comprises an ultrasonic sensor.

4. The system of claim 2, wherein the input means is further configured to receive a selected mode of operation for execution of the therapeutic laser procedure based on anatomy at the surgical site.

5. The system of claim 2, wherein the parameter associated with the laser source comprises at least one of a laser pulse frequency, a laser pulse energy or a laser pulse duration.

6. The system of claim 2, wherein the evaluation unit determines the temperature based at least in part on an equation:
$$T = ( 14.4 K * \frac{[ Laser power [ W ] ]}{[ Flushing flow rate [ ml / min ] ]} ) + Flushing fluid temperature [ K ]$$

7. The system of claim 2, wherein the user perceptible feedback comprises at least one of haptic, acoustic, or visual perceptible feedback.

8. The system of claim 2, wherein the flushing fluid is supplied from a flushing fluid reservoir to the surgical site via a flushing fluid channel, the system further comprising a cooling unit that is (i) coupled to at least one of the flushing fluid channel or the flushing fluid reservoir, and (ii) activated or controlled by the comparator unit.

9. The system of claim 2, wherein the flushing fluid is supplied from a flushing fluid reservoir to the surgical site via a flushing fluid channel, the system further comprising a filling-level sensor for detecting a filling level of the flushing fluid in the flushing fluid reservoir.

10. The system of claim 9, wherein the filling-level sensor is provided on the flushing fluid reservoir and/or is a software-based evaluation algorithm implemented in a computer unit, the computer unit determining the filling level based at least in part on the determined flow rate and a measured time period during which a flushing fluid discharge takes place.

11. The system of claim 10, wherein the computer unit is further configured to (i) predict a time period until complete emptying of the flushing fluid reservoir and/or (ii) generate a warning signal when a minimum remaining quantity of the flushing fluid is reached.

12. The system of claim 2, further comprising a flushing fluid delivery unit for regulating the flow rate of the flushing fluid based at least in part on the determined temperature and/or the received value of the parameter associated with the laser source.

13. The system of claim 2, wherein the comparator unit is further configured to activate or deactivate an emergency shut-down mechanism.

14. The system of claim 2, wherein at least one of the flow sensor or the temperature sensor comprises a retrofit kit for attachment to an endoscope.

15. The system of claim 14, wherein the endoscope has an extracorporeal region that remains external to a patient during the therapeutic laser procedure, and at least one of the temperature sensor or the flow sensor is mounted in the extracorporeal region of the endoscope.

16. A method of providing user perceptible feedback during a therapeutic laser procedure, the method comprising:
determining a flushing flow rate of a flushing fluid supplied to a surgical site;
receiving a value of a parameter of a laser system such that a laser power can be applied at a location of a light emission aperture;
determining a temperature of the flushing fluid;
computationally determining a temperature at the location of the light emission aperture during the therapeutic laser procedure based at least in part on the determined flushing flow rate, the determined temperature of the flushing fluid, and a value of the parameter of the laser system; and
providing the user perceptible feedback when (i) the determined temperature reaches or exceeds a first predetermined threshold, and/or (ii) the determined temperature falls below a second predetermined threshold.

17. The method of claim 16, further comprising selecting a mode of operation for execution of the therapeutic laser procedure based on anatomy at the surgical site.

18. The method of claim 16, wherein the parameter of the laser system comprises at least one of a laser pulse frequency, a laser pulse energy or a laser pulse duration.

19. The method of claim 16, wherein the temperature is determined based on an equation:
$$T = ( 14.4 K * \frac{[ Laser power [ W ] ]}{[ Flushing flow rate [ ml / min ] ]} ) + Flushing fluid temperature [ K ]$$

20. The method of claim 16, wherein the user perceptible feedback comprises at least one of haptic, acoustic, or visual perceptible feedback.

21. The method of claim 16, further comprising actively cooling the flushing fluid.

22. The method of claim 16, further comprising detecting a filling level of the flushing fluid in a flushing fluid reservoir.

23. The method of claim 22, further comprising (i) predicting a time period until complete emptying of the flushing fluid reservoir and (ii) generating a warning signal when a minimum remaining quantity of the flushing fluid is reached.

24. The method of claim 16, further comprising regulating the flow rate of the flushing fluid based at least in part on the determined temperature and/or the received value of the parameter of the laser system.
